(19) United States
(12) Patent Application Publication
(10) Pub. No.: US 2007/0293887 A1
(43) Pub. Date: Dec. 20, 2007

(54) Catheter assembly

(75) Inventors: Naohisa Okushi, Shizuoka (JP); Kinya Harada, Shizuoka (JP)

Correspondence Address: BUCHANAN, INGERSOLL & ROONEY PC, POST OFFICE BOX 1404, ALEXANDRIA, VA, 22313-1404, US

(73) Assignee: Terumo Kabushiki Kaisha, Tokyo (JP)

(21) Appl. No.: 11/812,253

(22) Filed: Jun. 15, 2007

(30) Foreign Application Priority Data
Jun. 20, 2006 (JP) 2006-170706
Mar. 2, 2007 (JP) 2007-53411

(51) Int. Cl. A61M 29/00

(52) U.S. Cl. 606/200

(57) ABSTRACT

A catheter assembly comprises an elongated tubular outer catheter opening at its distal end, an elongated tubular intermediate catheter opening at its distal end and positioned in the outer catheter, an elongated inner catheter positioned in the intermediate catheter, and a head section provided at a distal part of the inner catheter. A deformable section serving as a stirring mechanism is provided to stir an atheroma. In use of the catheter assembly, at the time of capturing an atheroma, the deformable section is operated to stir the atheroma, and thereafter the head section slides inside the outer catheter so that the atheroma stirred by the deformable section is sucked in through the distal end of the outer catheter.

### TECHNICAL FIELD

The disclosure generally relates to a catheter. More specifically, the disclosure pertains to a catheter assembly to be inserted into an organism (living body) to capture foreign matter present in the organism.

### BACKGROUND DISCUSSION

Removal of a comparatively soft thrombus, or atheroma, generated in a blood vessel is conducted by use of a catheter assembly for sucking the atheroma. An example of a suction catheter device for this purpose is one described in U.S. Pat. No. 5,569,204.

The suction catheter device includes an outer catheter, an inner catheter (middle catheter) inserted in the outer catheter, and a syringe connected to a hub of the inner catheter. In sucking an atheroma present in a blood vessel by use of the suction catheter device configured as above, first, the outer catheter and the inner catheter are inserted into the blood vessel, and the distal opening of the inner catheter is located in the atheroma (or in the vicinity of (proximity to) the atheroma). Next, under this condition, the syringe is operated, i.e., a pusher (plunger) of the syringe is moved in the direction of the proximal end (in the proximal direction) relative to a syringe outer tube. This reduces the pressure inside the inner catheter, whereby the atheroma is sucked through the distal opening of the inner catheter. However, this suction catheter device suffers from the disadvantage that, in the case where the atheroma has a comparatively high viscosity, the atheroma cannot be brought into the inner catheter, i.e., the atheroma cannot be sucked inward even when the syringe is operated.

In addition, the inner catheter of the suction catheter device is in the shape of a long tube and so a comparatively large pressure resistance is generated when the syringe is operated. Due to the pressure resistance, it is difficult to operate the syringe, namely to operate the pusher (plunger) of the syringe, and it is virtually impossible to achieve assured suction of the atheroma.

Also, since the pressure resistance induces a pressure loss in the inner catheter, assured (satisfactory) suction of the atheroma cannot be attained even if the syringe is operated.

### SUMMARY

A catheter assembly to be inserted into an organism to capture foreign matter present in the organism comprises an elongated tubular outer catheter body possessing an open distal end, an elongated tubular intermediate catheter body positioned inside the outer catheter body and possessing an open distal end, an elongated inner filamentous element positioned inside the intermediate catheter body, with the inner filamentous element possessing a distal part, and an elastic head section at the distal part of the inside filamentous element and connected to the inner filamentous element, with the elastic head section protruding distally beyond the open distal end of the intermediate catheter body. Also included is stirring means insertable into the foreign matter for stirring the foreign matter as the stirring means protrudes distally beyond the distal open end of the outer catheter body. After stirring by the stirring means, the intermediate catheter body and the inner filamentous element are collectively moved in a proximal direction relative to the outer catheter body to move the head section in the proximal direction into the outer catheter body to suck the stirred foreign matter through the distal open end of the outer catheter body and into the outer catheter body.

The catheter assembly causes the foreign matter to be assuredly stirred by the stirring means, whereby the foreign matter is made comparatively soft (comparatively low in viscosity) before being sucked. This helps ensure that the foreign matter present in an organism can be reliably captured and removed, irrespective of the viscosity of the foreign matter.

In addition, in the case where the stirring means includes a deformable section adapted to be expanded/contracted, the foreign matter can be stirred more securely and thereby brought into the state of being easier to suck.

In addition, if the deformable section is provided with radiopacity, the deformed state (expanded state/contracted state) of the deformable section can be checked or confirmed under fluoroscopy.

An expansion amount restricting means can be provided to restrict the maximum amount of expansion of the deformable section to thereby inhibit or prevent the deformable section from being excessively expanded. This facilitates to again contract the deformable section after an excessive expansion of the deformable section is brought about.

With the sucking operation being conducted in the vicinity of the foreign matter, generation of a pressure loss in the outer catheter body can be avoided or prevented (restrained). Therefore, the foreign matter stirred by the stirring means can be sucked into the outer catheter body more assuredly. In addition, since the generation of a pressure loss, namely, a pressure resistance is prevented (restrained), the intermediate catheter body and the inner filamentous element can be collectively moved easily in the direction of the proximal end (in the proximal direction) relative to the outer catheter body.

By providing the inner filamentous element with a first lumen and the head section with a second lumen, insertion/non-insertion of the guide wire in these lumens can be selected. This makes it possible to select closure/opening of the second lumen and, hence, to suck the foreign matter in the condition where the second lumen is closed.

According to another aspect, a catheter assembly to be inserted into an organism to capture foreign matter present in the organism comprises an elongated tubular outer catheter body possessing a lumen opening at a distal end of the outer catheter body, an elongated tubular intermediate catheter body positioned in the lumen of the outer catheter body and possessing a lumen opening at a distal end of the intermediate catheter body, with the intermediate catheter being longitudinally movable relative to the outer catheter body, an elongated tubular inner catheter body positioned in the lumen of the intermediate catheter body, with the inner catheter being longitudinally movable relative to the outer catheter body, and a tube connected to the intermediate catheter body and the inner catheter body at respective spaced apart locations. An intermediate portion of the tube between the two spaced apart locations comprises a deformable section that is positionable in the foreign matter and deformable upon application of a force to the deformable section. The inner catheter body and the intermediate catheter body are relatively longitudinally movable to apply a force to the deformable section of the tube positioned in the foreign matter to alternatively expand and contract the deformable section in a manner which stirs the foreign matter, with the stirred foreign matter being subsequently capturable in the outer catheter body.

In accordance with another aspect, a catheter assembly to be inserted into an organism to capture foreign matter present in the organism comprises an elongated tubular outer catheter body possessing a lumen opening at a distal end of the outer catheter body, with the lumen possessing a diameter and defining an inner surface of the outer catheter body, an elongated tubular intermediate catheter body positioned in the lumen of the outer catheter body and possessing a lumen opening at a distal end of the intermediate catheter body, with the intermediate catheter being longitudinally movable relative to the outer catheter body, and an elongated tubular inner catheter body positioned in the lumen of the intermediate catheter body, with the inner catheter being longitudinally movable relative to the outer catheter body. A head section is connected to the intermediate catheter body to move together with the intermediate catheter body. The head section is comprised of elastic material and protrudes distally beyond the distal end of the outer catheter body in a normal state of the head section. The head section is movable into the outer catheter body by longitudinal movement of the intermediate catheter body in a proximal direction relative to the outer catheter body. The head section possesses a maximum outer dimension portion at which an outer dimension of the head section is greatest. The maximum outer dimension portion of the head section is structurally sized relative to the diameter of the lumen in the outer catheter body such that when the head section is moved into the lumen of the outer catheter body from the normal state as a result of longitudinal movement of the intermediate catheter body in the proximal direction relative to the outer catheter body, the maximum outer dimension portion of the head section engages the inner surface of the outer catheter body and creates a space of reduced pressure in the lumen of the outer catheter body distally of the head section to draw the foreign matter into the lumen of the outer catheter body.

Other aspects pertain to a method of removing foreign matter present in an organism.

### BRIEF DESCRIPTION OF THE DRAWING FIGURES

FIG. 1 is an exploded view, partly in longitudinal cross-section, of a first embodiment of the catheter assembly disclosed herein.

FIG. 2 is an enlarged longitudinal cross-sectional view of a portion of the catheter assembly shown in FIG. 1 illustrating one of the sequential positions of the catheter assembly during use.

FIG. 3 is an enlarged longitudinal cross-sectional view of a portion of the catheter assembly illustrating another of position of the catheter assembly during use.

FIG. 4 is an enlarged longitudinal cross-sectional view of a portion of the catheter assembly illustrating the catheter assembly in another of the sequential positions during use.

FIG. 5 is an enlarged longitudinal cross-sectional view of a portion of the catheter assembly illustrating another sequential position of the catheter assembly during use.

FIG. 6 is an enlarged longitudinal cross-sectional view of a portion of the catheter assembly shown in FIG. 1 illustrating a further sequential position of the catheter assembly during use.

FIG. 7 is a longitudinal sectional view showing a configuration example of inserted condition maintaining means of the catheter assembly shown in FIG. 1;

FIG. 8 is a longitudinal cross-sectional view of one example of deformed condition maintaining means in the catheter assembly.

FIG. 9 is a partial longitudinal cross-sectional view showing a second embodiment of the catheter assembly.

FIG. 10 is an enlarged longitudinal cross-sectional view of a portion of a catheter assembly according to a third embodiment illustrating one of the sequential positions of the catheter assembly during use.

FIG. 11 is an enlarged longitudinal cross-sectional view of a portion of the catheter assembly shown in FIG. 10 illustrating another of the sequential positions of the catheter assembly during use.

FIG. 12 is an enlarged longitudinal cross-sectional view of a portion of the catheter assembly shown in FIG. 10 illustrating another of the sequential positions of the catheter assembly during use.

FIG. 13 is an enlarged longitudinal cross-sectional view of a portion of a catheter assembly according to a fourth embodiment illustrating one of the sequential positions of the catheter assembly during use.

FIG. 14 is an enlarged longitudinal cross-sectional view of a portion of the catheter assembly shown in FIG. 13 illustrating another of the sequential positions of the catheter assembly during use.

FIG. 15 is an enlarged longitudinal cross-sectional view of a portion of the catheter assembly shown in FIG. 13 illustrating another of the sequential positions of the catheter assembly during use.

FIG. 16 is an enlarged view showing the vicinity of an inside hub of the catheter assembly shown in FIGS. 13 to 15.

FIG. 17 is a partly longitudinally cross-sectional view showing the vicinity of the inside hub of a fifth embodiment of the catheter assembly.

FIG. 18 is an enlarged longitudinal cross-sectional view (a view showing a contracted state) showing a deformed state of a deformable section of a sixth embodiment of the catheter assembly disclosed herein.

FIG. 19 is an enlarged longitudinal cross-sectional view (a view showing a maximally expanded state) showing a deformed state of the deformable section of the catheter assembly shown in FIG. 18.

### DETAILED DESCRIPTION

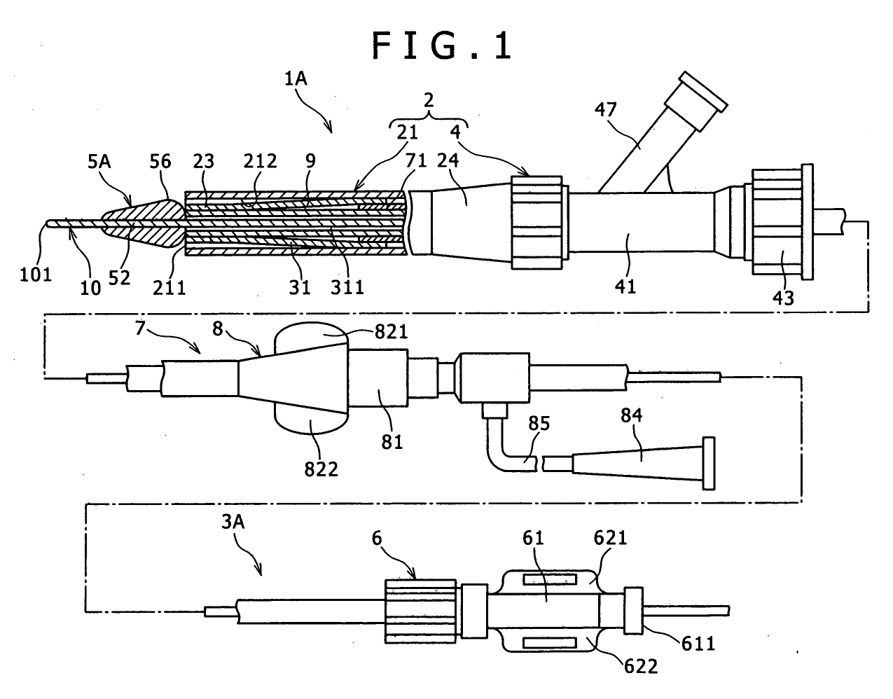

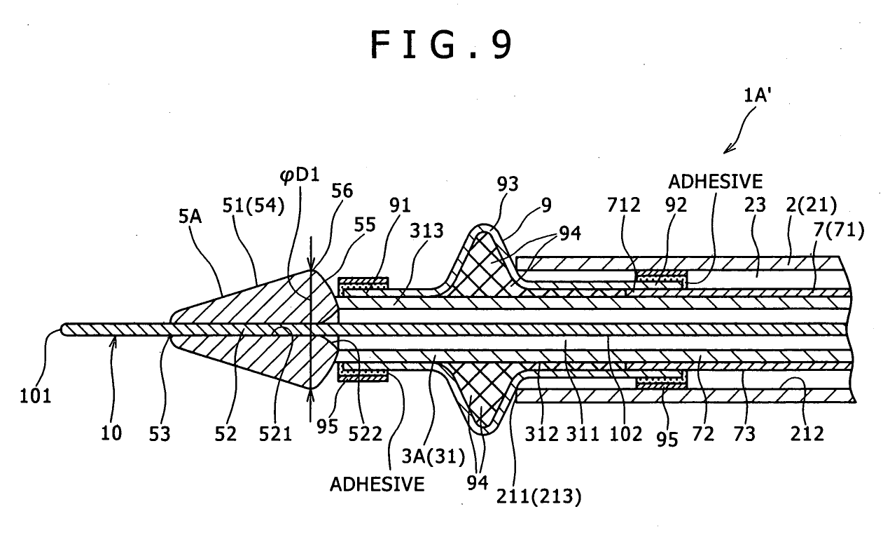

FIGS. 1-8 illustrate one embodiment of the catheter assembly disclosed herein. For convenience of description, the right side in FIGS. 1 to 8 (also in FIGS. 9 to 19) will be referred to as the “proximal end,” and the left side as the “distal end.”

The catheter assembly 1A shown in FIG. 1 is intended to be inserted into an organism, such as a blood vessel, to capture and remove foreign matter present in the organism. One example of foreign matter to be captured and removed (sucked and removed) by the catheter assembly 1A is a thrombus 200 having a comparatively high viscosity (being gruel-like or jellylike) generated in a blood vessel (such a thrombus will hereinafter be referred to as “atheroma”).

The catheter assembly 1A includes an outer catheter 2, an intermediate catheter 7, an inner catheter (inner structure) 3A and a deformable section 9 (an example of a stirring means). The catheter assembly 1A is used in combination with a guide wire 10. Specifically, as shown in FIG. 1, the catheter assembly 1A is used in the condition (inserted condition) where the intermediate catheter 7 is inserted in the outer catheter 2, the inner catheter 3A is inserted in the intermediate catheter 7, and further the guide wire 10 is inserted in the inner catheter 3A.

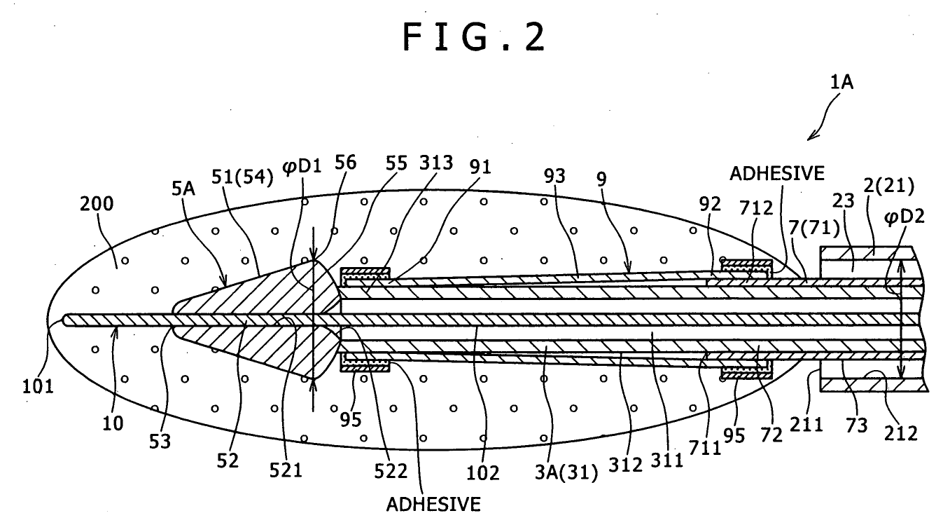

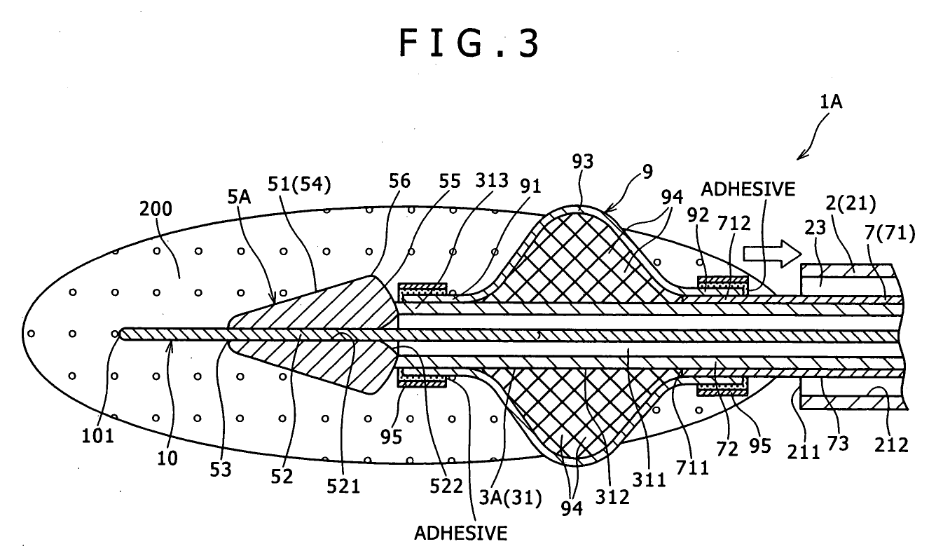

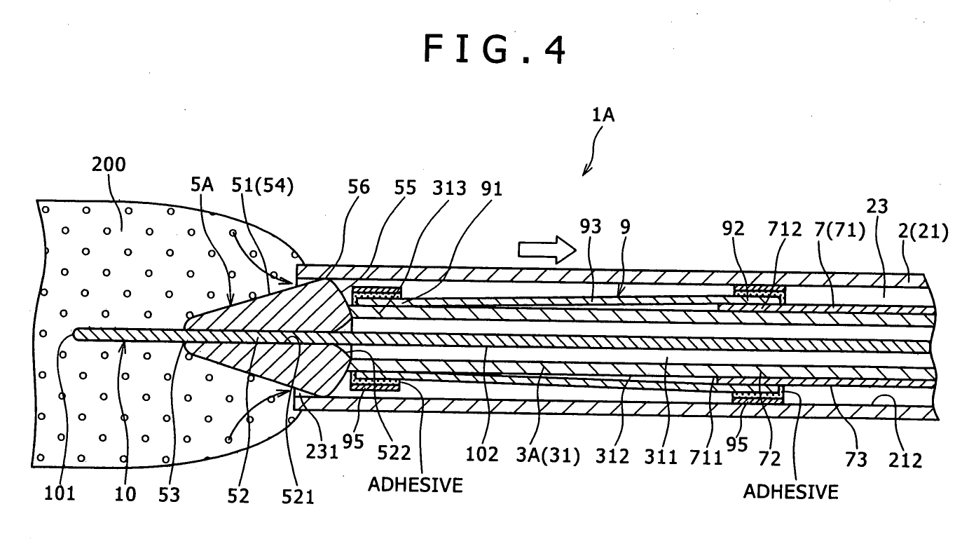

In the catheter assembly 1A configured in this way, at the time of capturing the atheroma 200, the atheroma 200 is first stirred (as generally indicated in FIGS. 2 and 3). Thereafter, the atheroma 200 thus stirred so as to be comparatively soft is sucked (as generally illustrated in FIGS. 4 to 6). The stirring operation is conducted by an operation involving moving (reciprocating) the inner catheter 3A (inner catheter body or inner filamentous element 31) in its longitudinal direction relative to the intermediate catheter 7 (intermediate catheter body 71). In addition, the sucking operation is conducted by moving the intermediate catheter 7, the inner catheter 3A and the guide wire 10 collectively in the direction of the proximal end (in the proximal direction) relative to the outer catheter 2.

Before describing the catheter assembly 1A according to this disclosed embodiment, the guide wire 10 to be inserted in the inner catheter 3A will be described.

The guide wire 10 shown in FIGS. 1 to 6 is a flexible filamentous body. Examples of the material constituting the guide wire 10 include various metallic materials such as stainless steels, cobalt-based alloys, alloys exhibiting superelasticity (inclusive of superelastic alloys), and piano wire.

The distal surface or distal end 101 of the guide wire 10 is rounded. This enables smooth insertion (movement) of the guide wire 10 in the distal direction in the blood vessel. Also, the distal surface 101 can be reliably prevented from damaging the blood vessel wall when the guide wire 10 is moved toward the distal direction (moved forward) in the blood vessel.

Now, the various component sections of the catheter assembly 1A will be described.

As shown in FIG. 1, the outer catheter 2 includes an elongated tubular outer catheter body 21, and an outside hub 4 connected to a proximal end of the outer catheter body 21.

The outer catheter body 21 has desired flexibility. Examples of the material constituting the outer catheter body 21 include polyolefins such as polyethylene, polypropylene, polystyrene, polyamides, polyimides, polyether-ether ketones, polyurethane, polyesters such as polyethylene terephthalate, polybutylene terephthalate, fluororesins such as polytetrafluoroethylene, various thermoplastic elastomers based on polyolefin, polystyrene, polyamide, polyurethane, polyester, fluororubber, chlorinated polyethylene, and combinations (polymer alloys, polymer blends) of two or more of these materials.

In addition, the outer catheter body 21 may have a multilayer laminate structure composed of a plurality of kinds of materials. Also, a reinforcement such as a braid and/or coil may be embedded in the outer catheter body 21.

In addition, the outer catheter body 21 is preferably sufficiently transparent to enable visual checking of the sucked atheroma 200 through the outer catheter body 21.

The outer catheter body 21 is formed therein with a lumen 23 along its longitudinal direction. The lumen 23 opens to the distal end (distal opening) 211 of the outer catheter body 21. The lumen 23 can be used for insertion of the intermediate catheter 7 (intermediate catheter body 71) and a head section (tip) 5A of the inner catheter 3A described later, and can also be used to supply a liquid medicine or the like into the blood vessel or for sucking (drawing inward) a liquid.

The surface defining the lumen 23, i.e., the inner peripheral surface 212 of the outer catheter body 21, may be provided with a coating layer for reducing the frictional resistance between the interior surface of the outer catheter body and the outer peripheral surface 51 of the head section 5A of the inner catheter 3A. That is, the surface defining the lument 23 may be subjected to a friction reducing treatment. This enables smoother insertion and evulsion of the inner catheter 3A (which is inserted in the intermediate catheter 7) relative to the outer catheter 2. This helps ensure that the sucking operation for the atheroma 200 (i.e., the drawing operation in which the atheroma is drawn in) is capable of being performed more securely or reliably, and the atheroma 200 can be sucked more assuredly by the sucking operation. Examples of the coating layer include a coating layer of a fluororesin such as polytetrafluoroethylene (“Teflon” coating (“Teflon” is a registered trademark)), a silicone coating, and a hydrophilic polymer coating exhibiting a lubricating property when wetted.

In addition, at least in the vicinity of the distal end 211 of the outer catheter body 21, there may be provided a member (e.g., a ring-like (tubular) or coil-like member) formed of a material (e.g., platinum, gold, tungsten) having radiopacity (radiographic contrast property). This permits visual checking or confirmation of the location of the distal end 211 of the outer catheter body 21 under fluoroscopy.

Also, a coating member (anti-kink protector) 24 which provides reinforcement is located at the proximal end of the outer catheter body 21, i.e., at a part of the outer catheter body 21 connected to the outside hub 4. This makes it possible to more effectively prevent this part from kinking. The material constituting the coating member 24 is not particularly limited. Examples of suitable materials include polyolefins, polyamides, polyesters such as polyethylene terephthalate, polybutylene terephthalate, polyurethane, polyvinyl chloride, ABS resin, AS resin, fluororesin such as polytetrafluoroethylene, and various thermoplastic elastomers such as polyamide elastomers, polyester elastomers, polyurethane elastomer.

In addition, the length of the outer catheter body 21 is not particularly limited. For example, the length is preferably 300 to 1,800 mm, more preferably 600 to 1,400 mm.

The outside diameter of the outer catheter body 21 is also not specifically limited to a certain value. As an example, the outside diameter is preferably 1.0 to 6.0 mm, more preferably 1.5 to 4.5 mm.

Similarly, the inside diameter (φD2) of the outer catheter body 21 is not particularly limited. For example, the inside diameter is preferably 1.0 to 4.0 mm, more preferably 1.2 to 3.5 mm.

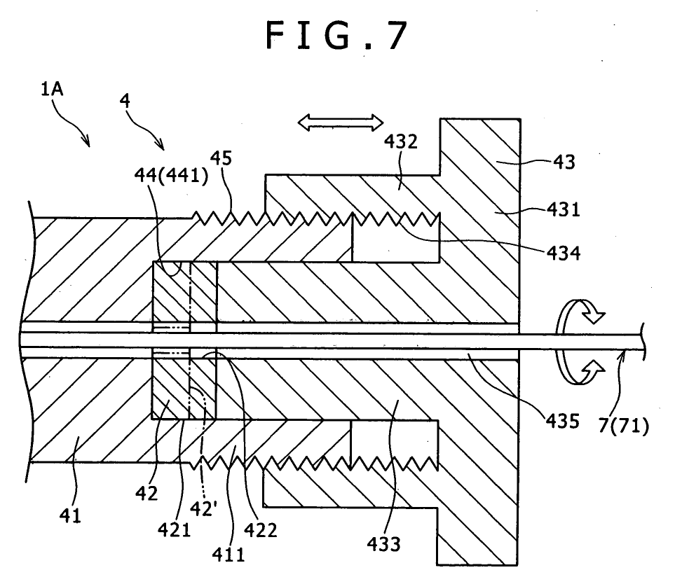

The outside hub 4 is connected in a liquid-tight manner to the proximal end of the outer catheter body 21. The outside hub 4 includes a tubular outside hub body 41, a branch section 47 branching from an intermediate part of the outside hub body 41, a ring-like member 42 (shown in FIG. 7) contained in the outside hub body 41, and a pushing member 43 for pushing the ring-like member 42.

The outside hub body 41 has an inside space communicating with the lumen 23 of the outer catheter body 21.

The branch section 47 is tubular and communicates with the outside hub body 41. Through the branch section 47, for example, a liquid medicine can be fed into the outer catheter 2, or the sucked (drawn-in) atheroma 200 can be removed.

As illustrated in FIG. 7, the proximal end 411 of the outside hub body 41 is provided with a cylindrical recess 44 extending along the longitudinal direction of the outside hub body 41. The diameter of the recess 44 is greater than the inside diameter of the tubular outside hub body 41. In addition, a male screw part 45 adapted to threadably engage the pushing member 43 is provided at the outer surface of the proximal end 411 of the outside hub body 41.

A ring-like member 42 is contained in the recess 44 of the outside hub body 41. The ring-like member 42 is formed of an elastic material. The ring-like member 42, in its natural state, has an inside diameter approximately equal to the inside diameter of the outside hub body 41, and an outside diameter approximately equal to the inside diameter of the recess 44. The term “natural state” herein means the condition where no external force is being exerted on the ring-like member 42.

The pushing member 43 includes a disk-like section 431, a tubular section 432 provided concentrically with the disk-like section 431, and a cylindrical section 433 provided concentrically with the disk-like section 431. Thus, the tubular section 432 and the cylindrical section 433 are concentric with one another.

The inner peripheral surface of the tubular section 432 is formed with a female screw part 434 for threaded engagement with the male screw part 45 at the outside hub body 41 (i.e., at the proximal end 411 of the outside hub body 41 in the illustrated embodiment). This enables the pushing member 43 to be rotated while being in screw engagement with the outside hub body 41.

The cylindrical section 433 has an outer diameter approximately equal to the inside diameter of the recess 44.

The pushing member 43 is further formed with a through-hole 435 penetrating the pushing member 43 from one end to the other along its longitudinal direction. The diameter of the through-hole 435 is approximately equal to the inside diameter of the outside hub body 41.

As the pushing member 43 and the outside hub body 41 are screw engaged with each other, the ring-like member 42 is pushed by the distal face of the cylindrical section 433 of the pushing member 43. Under this pushing force, the ring-like member 42 tends to increase in outside diameter through elastic deformation. However, since the outer peripheral surface 421 of the ring-like member 42 is restricted by the inner peripheral surface 441 of the recess 44, the ring-like member 42 is prevented from increasing in outside diameter. Therefore, the inside diameter of the ring-like member 42 is reduced (see the reduced inside diameter state of the ring-like member 42′ illustrated in FIG. 7). As a result, the intermediate catheter body 71 inserted in the outside hub body 41 is pressed (compressed) by the inner peripheral surface 422 of the ring-like member 42 so that the intermediate catheter body 71 is reliably fixed.

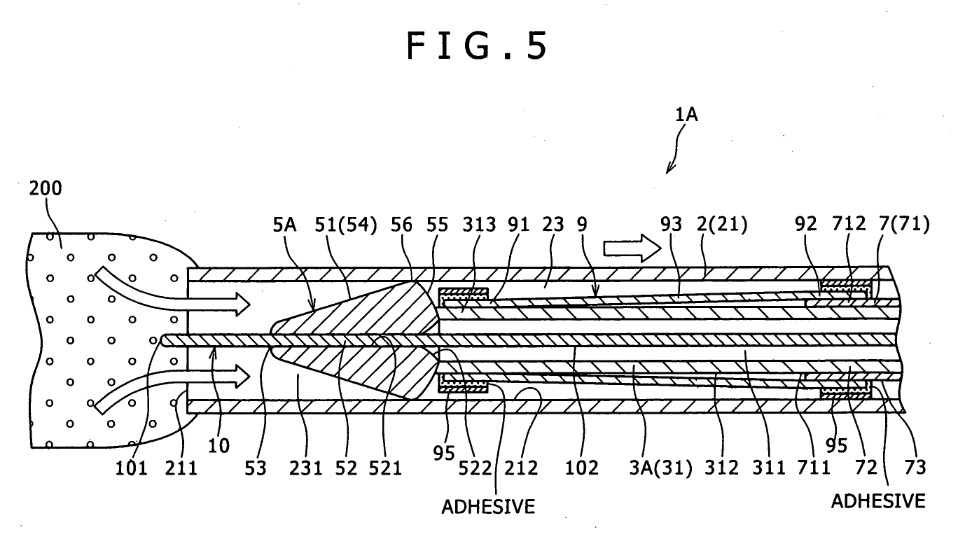

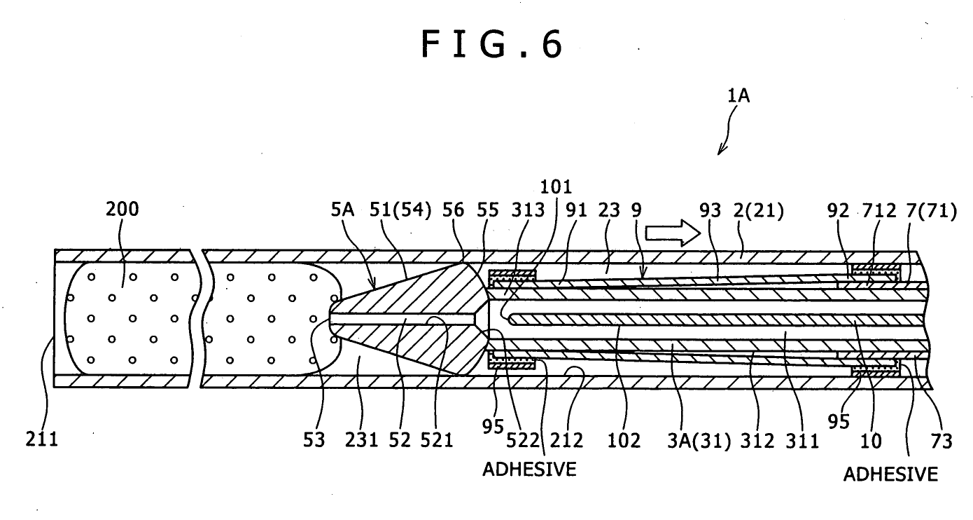

Thus, in the catheter assembly 1A of the illustrated embodiment, the recess 44 in the outside hub body 41, the ring-like member 42 and the pushing member 43 constitute one example of an inserted condition maintaining means (fixing means) for fixing the intermediate catheter 7. By virtue of the inserted condition maintaining means, it is possible to securely maintain the assembled condition, i.e., the condition in which the intermediate catheter 7 is inserted in (positioned inside of) the outer catheter 2. In this illustrated embodiment, the assembled condition can include the condition where the head section 5A of the inner catheter 3A protrudes from the outer catheter 2 as shown in FIG. 1, the condition where the head section 5A and the deformable section 9 protrude from the outer catheter 2 as shown in FIG. 2 (FIG. 3), and the condition where the head section 5A is contained (located) in the outer catheter 2 as shown in FIG. 6 (and also in FIG. 4 and in FIG. 5). In this embodiment of the catheter assembly 1A, the inserted condition maintaining means makes it possible to maintain the condition shown in FIG. 1, the condition shown in FIG. 2, and the condition shown in FIG. 6.

Depending on the extent of fixation, i.e., depending on the pushing amount of the pushing member 43, the inserted condition maintaining means can securely fix collectively the intermediate catheter 7 and the inner catheter 3A inserted in the intermediate catheter 7. Further, by increasing the extent of fixation, the inserted condition maintaining means can securely fix collectively the intermediate catheter 7, the inner catheter 3A inserted in the intermediate catheter 7, and the guide wire 10 inserted in the inner catheter 3A.

The material constituting the outside hub 4 (exclusive of the ring-like member 42) is not particularly limited. As examples, various metallic materials, various plastics and the like can be used, either singly or in combination, as the material for forming the outside hub 4.

In addition, the elastic material constituting the ring-like member 42 is not particularly limited. Examples of possible materials which can be used to form the ring-like member 42 include various rubber materials (particularly, vulcanized rubber materials) such as natural rubber, isoprene rubber, butadiene rubber, styrene-butadiene rubber, nitrile rubber, chloroprene rubber, butyl rubber, acrylic rubber, ethylene-propylene rubber, hydrin rubber, urethane rubber, silicone rubber, fluororubber and various thermoplastic elastomers based on styrene, polyolefin, polyvinyl chloride, polyurethane, polyester, polyamide, polybutadiene, trans-polyisoprene, fluororubber, chlorinated polyethylene or the like, which may be used either singly or in combination of two or more of them.

The intermediate catheter 7 includes the elongated tubular intermediate catheter body 71, and an intermediate hub 8 connected to a proximal end of the intermediate catheter body 71.

The intermediate catheter body 71 has desirable flexibility characteristics and is inserted in the outer catheter body 21. By way of example, the intermediate catheter body 71 can be made of the same materials as the above-mentioned materials for the outer catheter body 21. Among these materials, polyimides and polyether-ether ketones are preferred due to their hardness characteristics and their respective modulus of elasticity.

In addition, the intermediate catheter body 71 may have a multilayer laminate structure composed of a plurality of materials.

A lumen 72 is provided in the intermediate catheter body 71 and extends along the longitudinal direction of the intermediate catheter body 71 from one end to the other. The lumen 72 opens to the distal end (distal opening) 711 of the intermediate catheter body 71. The lumen 72 is used for insertion of the inner catheter 3A (inner catheter body 31) therein.

The outside diameter of the intermediate catheter body 71 is not limited to any specific dimension. By way of example, the outside diameter is preferably 0.9 to 3.0 mm, more preferably 1.1 to 2.7 mm.

Similarly, the inside diameter of the intermediate catheter body 71 is not particularly limited. For example, the inside diameter is preferably 0.8 to 2.8 mm, more preferably 0.9 to 2.5 mm.

Also, the length of the intermediate catheter body 71 can vary. As an example, the length is preferably 350 to 1,750 mm, more preferably 450 to 1,650 mm.

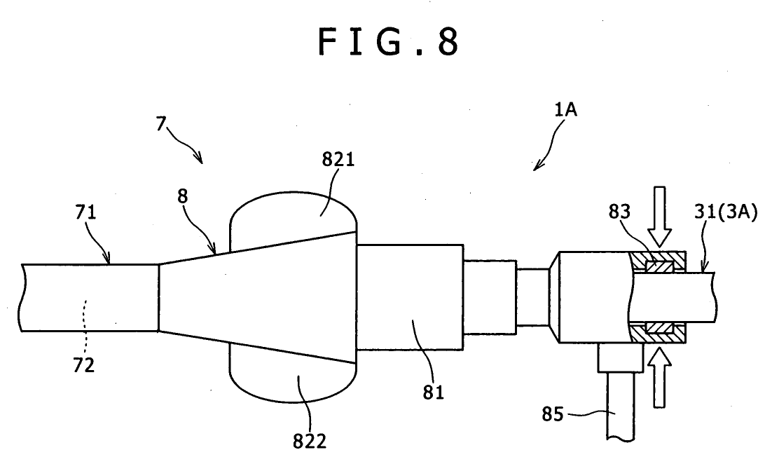

The intermediate hub 8 is connected in a liquid-tight manner to the proximal end of the intermediate catheter body 71 by, for example, adhesion (adhesion by use of an adhesive or a solvent). The intermediate hub 8 includes a tubular intermediate hub body 81, a pair of wing sections 821, 822 projecting outwardly from the outer peripheral surface of the intermediate hub body 81, a valve element 83, a connector 84, and a tube 85 connecting the connector 84 and the intermediate hub body 81 to each other as shown in FIGS. 1 and 8.

The intermediate hub body 81 has an inside space communicating with the lumen 72 of the intermediate catheter body 71.

On the upper side and the lower side in FIG. 1, and in FIG. 8, of the distal part of the intermediate hub body 81, the wing sections 821, 822 are integrally formed in one piece with the intermediate hub body 81. The wing sections 821, 822 are composed of small pieces. When the intermediate catheter 7 is moved relative to the outer catheter 2, the operation can be relatively easily performed by holding the wing sections 821, 822.

As shown in FIG. 8, the valve element 83 formed of an elastic material is press fitted into the proximal end portion of the intermediate hub body 81. The valve element 83 is ring-like in shape so that the inner catheter body 31 can be inserted in the inside of the valve element 83.

In the assembled condition, the valve element 83 compresses the inner catheter body 31 in the radial direction (the direction of the arrows in FIG. 8). This maintains the liquid-tightness inside the intermediate hub body 81. In addition, the inner catheter 3A (inner catheter body 31) can be fixed at an arbitrary or desired position relative to the intermediate catheter 7 (intermediate hub 8).

The connector 84 is connected to an intermediate part of the intermediate hub body 81 through the tube 85. The connector 84 is so configured that a syringe filled with a liquid, such as a liquid medicine, can be connected in a liquid-tight manner thereto. In the condition where the syringe is connected to the connector 84, a liquid can be supplied from the syringe into the intermediate catheter body 71. In other words, priming can be performed.

Incidentally, the material constituting the intermediate hub 8 (exclusive of the valve element 83) is not particularly limited. For example, like in the case of the above-described outside hub 4, various metallic materials and various plastics can be used, either singly or in combination with one another, as the material for forming the intermediate hub 8. When such a material is used, the intermediate hub 8 is comparatively hard, so that it is possible, for example, to easily insert the guide wire 10 into the inner catheter body 31 through the intermediate hub 8. In addition, the elastic material forming the valve element 83 can be, for example, the same materials as those usable for the ring-like member 42 of the outer catheter 2.

As shown in FIG. 1, the inner catheter 3A includes an elongated filamentous element forming an elongated tubular inner catheter body 31, a head section 5A as a sucking body provided at the distal end of the inner catheter body 31, and the inside hub 6 connected to a proximal end of the inner catheter body 31.

The inner catheter body 31 has desirable flexibility characteristics, and is inserted in the intermediate catheter body 71. Examples of the material forming the inner catheter body 31 include substantially the same materials as those usable for the outer catheter body 21 mentioned above. Among the possible materials, polyimides and polyether-ether ketones are preferred, in view of their hardness characteristics and respective modulus of elasticity characteristics.

The inner catheter body 31 may have a multilayer laminate structure composed of a plurality of materials. In addition, a reinforcement such as a braid and/or coil may be embedded in the inner catheter body 31.

A first lumen 311 extends within the inner catheter body 31 from one end to the other along the longitudinal direction of the inner catheter body 31. The first lumen 311 can function not only as an inserting passage in which to insert the guide wire 10, but also as a supplying passage for supplying a liquid medicine or the like into a blood vessel.

In addition, the outside diameter of the inner catheter body 31 is set to be smaller than the inside diameter of the intermediate catheter body 71. That is, in the assembled condition, a gap is generated between the outer peripheral surface 312 of the inner catheter body 31 and the inner peripheral surface of the intermediate catheter body 71. This makes it possible to smoothly move the inner catheter 3A relative to the intermediate catheter 7 and, hence, to stir the atheroma 200 more assuredly (see FIGS. 2 and 3). Although the outside diameter of the inner catheter body 31 is not particularly limited, by way of example, the outside diameter is preferably 0.5 to 2.5 mm, more preferably 0.7 to 2.3 mm.

The inside diameter of the inner catheter body 31 is also not limited to a specified dimension. By way of example though, the inside diameter is preferably 0.3 to 2.3 mm, more preferably 0.5 to 2.1 mm.

In addition, the length of the inner catheter body 31 is not particularly limited. For example, the length is preferably 450 to 1,850 mm, more preferably 550 to 1,750 mm.

As shown in FIGS. 1 to 6, the head section 5A which is elastic or compressible is joined to the distal end of the inner catheter body 31. The method of joining can take various forms. For example, adhesion (adhesion by use of an adhesive or a solvent) and fusing (heat fusing, high-frequency fusing, ultrasonic fusing, etc.) may be used for the joining.

The head section 5A is cylindrical in outer shape.

The head section 5A is formed therein with a second lumen 52 along its longitudinal direction. The second lumen 52 extends from one end of the head section to the other and communicates with the first lumen 311. In addition, the second lumen 52 opens at the distal end 53 of the head section 5A. Like the first lumen 311, the second lumen 52 thus configured can function not only as an inserting passage in which to insert the guide wire 10, but also as a passage for supplying a liquid medicine or the like into a blood vessel through the distal end 53 of the head section 5A.

In its natural state, the head section 5A has an inside diameter approximately equal to or slightly smaller than the outside diameter of the guide wire 10. This ensures that, as shown in FIG. 1 (and also in FIGS. 2-5), in the condition where the guide wire 10 is inserted in the inner catheter 3A, the inner peripheral surface 521 of the second lumen 52 of the head section 5A makes close contact with the outer peripheral surface 102 of the guide wire 10, i.e., the second lumen 52 is plugged up or closed by the guide wire 10.

In addition, the head section 5A includes a first taper section 54 and a second taper section 55. The outside diameter of the first taper section 54 gradually decreases along the distal direction. The second taper section 55 is located on the proximal end relative to the first taper section 54 and the outside diameter of the second taper section 55 gradually decreases along the proximal direction. As a result, the head section 5A has a maximum outside diameter part 56 representing a maximum outside diameter of the head section 5A at an intermediate part of the head section 5A, specifically, at the boundary between the first taper section 54 and the second taper section 55. When the head section 5A is in a natural state, the outside diameter (maximum outside diameter) φD1 (indicated in FIG. 2) of the maximum outside diameter part 56 is set to be approximately equal to or slightly smaller than the inside diameter φD2 (see FIG. 2) of the outer catheter body 21. This permits the head section 5A to slide in the outer catheter body 21 as generally indicated in FIGS. 4-6.

In the assembled condition, the head section 5A protrudes from the distal end 711 of the intermediate catheter 7. The outside diameter φD1 of the maximum outside diameter part 56 of the head section 5A is set to be greater than the outside diameter of the inner catheter body 31 and the inside diameter of the intermediate catheter body 71. This ensures that the head section 5A is prevented from entering into the intermediate catheter 7 and, therefore, the inner catheter 3A can be securely prevented from being excessively pulled (moved) to the proximal direction relative to the intermediate catheter 7.

When the inner catheter body 31 and the intermediate catheter body 71 are moved together with the guide wire 10 in the proximal direction relative to the outer catheter 2, starting from the condition shown in FIG. 2, i.e., the condition where the head section 5A protrudes from the distal end 211 of the outer catheter body 21 into the atheroma 200 in the assembled condition, the conditions shown in FIGS. 4-6 sequentially occur. That is, the head section 5A is pulled into the outer catheter 2 and is slid inside the outer catheter 2. Associated with this, a reduction in pressure is induced on the distal side, relative to the maximum outside diameter part 56, of the outer catheter body 21, i.e., the space 231 defined by the inner peripheral surface 212 of the outer catheter body 21 and the first taper section 54 (outer peripheral surface 51) of the head section 5A. The stirred atheroma 200 is assuredly sucked or drawn (contained) into the space 231 in which the reduction in pressure is induced.

In addition, since the second lumen 52 is plugged up with, or closed off by, the guide wire 10, the pressure-reduced condition of the space 231 is maintained when the head section 5A is pulled into the outer catheter 2.

Since the sucking operation in the catheter assembly 1A is conducted in the vicinity of the atheroma 200, a pressure loss can be inhibited or prevented (restrained) from being generated in the space 231. Therefore, the atheroma 200 can be securely sucked or drawn into the space 231. In addition, since the generation of a pressure loss, or pressure resistance, is inhibited or prevented (restrained), the operation of moving the inner catheter body 31 and the intermediate catheter body 71 collectively can be easily performed.

When the inner catheter 3A and the intermediate catheter 7 are operated collectively, mainly the maximum outside diameter part 56 is slid inside the outer catheter body 21, whereas sliding of the first taper section 54, the second taper section 55 and the outer peripheral surface 73 of the intermediate catheter body 71 is restrained. This helps ensure that the collective operation of the inner catheter 3A and the intermediate catheter 7, or the sucking operation, can be easily carried out and, hence, the atheroma 200 can be assuredly sucked or drawn in.

As has been described above, the head section 5A is provided with the first taper section 54. This helps ensure that, in the condition where the head section 5A protrudes from the distal end 211 of the outer catheter body 21, the head section 5A can be easily inserted into the atheroma 200 as shown in FIG. 2.

In addition, as mentioned above, the head section 5A is provided with the second taper section 55. This taper section helps ensure that, when the head section 5A protruding from the distal end 211 of the outer catheter body 21 enters into the outer catheter body 21, the entering movement is performed smoothly.

The inner peripheral surface 521 of the head section 5A is provided at its proximal end with an introducing section 522 possessing an inside diameter that gradually increases in the proximal direction. The guide wire 10 inserted via the proximal end opening 611 of the inside hub 6 of the inner catheter 3A passes through the first lumen 311, and is introduced into the second lumen 52 of the head section 5A along the introducing section 522.

The head section 5A is preferably formed of an elastic material. Examples of the elastic material include various rubber materials such as natural rubber, isoprene rubber, butadiene rubber, chloroprene rubber, silicone rubber, fluororubber, styrene-butadiene rubber and various thermoplastic elastomers based on styrene, polyolefin, polyurethane, polyester, polyamide, polybutadiene, trans-polyisoprene, fluororubber, chlorinated polyethylene or the like.

In addition, in the head section 5A, a material having radiopacity (e.g., platinum, gold, tungsten) may be contained in the elastic material. This enables the head section 5A to be assuredly checked or visually confirmed under fluoroscopy.

The outer peripheral surface 51 of the head section 5A may also be subjected to a friction reducing treatment, in the same manner as the inner peripheral surface 212 of the outer catheter body 21.

The length of the head section 5A can be various dimensions and is not particularly limited. By way of example, the length is preferably 5 to 15 mm, more preferably 8 to 12 mm.

The outside diameter φD1 of the maximum outside diameter part 56 of the head section 5A is also not particularly limited. For example, the outside diameter φD1 is preferably 1.0 to 3.5 mm, more preferably 1.3 to 3.3 mm.

In a similar manner, the inside diameter of the head section 5A is not particularly limited. By way of example, the inside diameter is preferably 0.5 to 1.5 mm, more preferably 0.7 to 1.3 mm.

In the illustrated and described embodiment, the head section 5A and the inner catheter body 31 are each hollow (are each provided with a lumen). However, this configuration is not required as these parts may, for example, be solid.

The inside hub 6 is connected in a liquid-tight manner to a proximal end of the inner catheter body 31. The inside hub 6 includes a tubular inside hub body 61, and a pair of wing sections 621, 622 projecting outwardly from the outer peripheral surface of the inside hub body 61.

The inside hub body 61 has an inside space communicating with the first lumen 311 of the inner catheter body 31.

The inside hub body 61 is provided, on the upper side and the lower side, in FIG. 1, with the wing sections 621, 622 which are integrally formed in one piece with the inside hub body 61. The wing sections 621, 622 are composed of small pieces. When the inner catheter 3A is moved relative to the intermediate catheter 7, the moving operation can be relatively easily performed by holding the wing sections 621, 622.

The material constituting the inside hub 6 is not limited to any particular material. Examples include various metallic materials and various plastics used either singly or in combination, in the same manner as in the case of the outside hub 4 above-mentioned. Where such a material is used, the inside hub 6 is comparatively hard so that it is possible, for example, to relatively easily insert the guide wire 10 into the inner catheter body 31 through the inside hub 6.

As shown in FIG. 1 (and also in FIGS. 2-6), the deformable section 9 is disposed on the distal side of the catheter assembly 1A. The deformable section 9 is an example of a stirring means for stirring the atheroma 200 at the time of capturing the atheroma 200.

The deformable section 9 is composed of a tube possessing a distal end 91 joined to a distal end 313 of the inner catheter body 31 and a proximal end 92 is joined to a distal end 712 of the intermediate catheter body 71. That is, the distal end 313 of the inner catheter body 31 and the distal end 712 of the intermediate catheter body 71 are couple to each other by the deformable section 9.

The method of joining the distal end 91 of the deformable section 9 to the distal end 313 of the inner catheter body 31 and the proximal end 92 of the deformable section 9 to the distal end 712 of the intermediate catheter body 71 is not limited to any specific method. For example, a method may be adopted in which, as shown in FIG. 3, as well as FIGS. 1, 2, 4-6, and 9-15, a respective tubular body 95 formed of a resin material or metallic material is fitted over each of the distal end 91 and the proximal end 92 of the deformable section 9, and the gap therebetween is filled with an adhesive.

In addition, each of the tubular bodies 95 may be provided with a coating layer for lessening (reducing) the frictional resistance between itself and the inner peripheral surface 212 of the outer catheter body 21.

The deformable section 9 thus disposed helps ensure that when the inner catheter body 31 is moved in the proximal direction (in the direction of the arrow in FIG. 3) relative to the intermediate catheter body 71 starting from the condition of protruding from the distal end 211 of the outer catheter body 21 (the condition shown in FIG. 2), the distal end 313 of the inner catheter body 31 approaches the distal end 712 of the intermediate catheter body 71, and a central part 93 of the deformable section 9 is expanded (enlarged in diameter) as shown in FIG. 3. In addition, when the inner catheter body 31 is moved in the distal direction relative to the intermediate catheter body 71 starting from the condition where the central part 93 of the deformable section 9 is expanded (the expanded condition shown in FIG. 3), the distal end 313 of the inner catheter body 31 is spaced away from the distal end 712 of the intermediate catheter body 71, and the central part 93 is contracted (reduced in diameter). That is, the deformable section 9 returns to the condition shown in FIG. 2 (the initial condition).

Thus, with the inner catheter body 31 reciprocated along its longitudinal direction relative to the intermediate catheter body 71, the deformable section 9 repeatedly undergoes expansion/contraction (deformation).

During use of the catheter assembly 1A, at the time of sucking and capturing an atheroma 200, first the atheroma 200 is stirred. The stirring is conducted by inserting the deformable section 9 in the initial condition into the atheroma 200 and operating the inner catheter body 31 in the above-mentioned manner. In other words, the stirring is conducted by inserting the deformable section 9 in the initial condition into the atheroma 200 and, in this inserted condition, operating the deformable section 9, i.e., repeatedly expanding/contracting the deformable section 9. With the atheroma 200 stirred, the viscosity of the atheroma 200 is lowered. That is, the atheroma 200 is softened. This helps ensure that the atheroma 200 can be reliably sucked or drawn in a sucking operation conducted after the stirring operation.

In addition, the deformable section 9 is provided with a multiplicity of through holes 94 by which the inside and the outside of the deformable section 9 communicate. That is, these holes 94 penetrate the wall part constituting the deformable section 9 as generally depicted in FIG. 3. The holes 94 may open when the deformable section 9 is expanded, or may open irrespective of the expansion/contraction of the deformable section 9.

With the deformable section 9 repeatedly brought into expansion/contraction in the state of being inserted in the atheroma 200, the atheroma 200 is caused to flow into the inside of the deformable section 9 through the holes 94, and the atheroma 200 which has been caused to flow into the inside of the deformable section 9 is caused to flow out to the outside through the holes 94. This enables more assured or reliable stirring of the atheroma 200.

In addition, the condition in which the deformable section 9 protrudes from the distal end 211 of the outer catheter body 21 by a predetermined projecting amount (for example, the deformable section 9 protrudes entirely as shown with reference to the disclosed embodiment in FIG. 2) is maintained by the above-mentioned inserted condition maintaining means. This helps ensure that, when it is desired to stir the atheroma 200, the deformable section 9 can be securely prevented from entering into the outer catheter 2 unwillingly.

As above-mentioned, the inner catheter body 31 for expansion/contraction of the deformable section 9 is fixed at an arbitrary position by the valve element 83 disposed in the intermediate hub 8 of the intermediate catheter 7. This makes it possible to securely maintain the expanded state and the contracted state of the deformable section 9 and, hence, to prevent the deformable section 9 from being expanded or contracted due to movement of the inner catheter body 31 unwillingly, i.e., without intended operation of the inner catheter body 31. As a result, the operation of capturing the atheroma 200 can be carried out relatively speedily.

Thus, the valve element 83 is an example of a deformed state maintaining means for maintaining the expanded state and the contracted state of the deformable section 9.

In addition, in this embodiment, the deformable section 9 is formed (configured) by combining a plurality of (e.g., eight or more) wires (filamentous elements) having a circular or polygonal cross-sectional shape in a net form, which net form is generally illustrated in FIG. 3. This helps enable the deformable section 9 to be deformed easily and assuredly.

The material constituting the deformable section 9 is preferably formed of an alloy exhibiting superelasticity in an organism (at least at an organism temperature (around 37□)) (hereinafter referred to as “superelastic alloy”), i.e., an alloy having a property such that even when the material formed in a phase (parent phase) is deformed in another phase, the alloy restores its original shape upon returning into the parent phase, namely, an alloy having a shape memory effect. Examples of the superelastic alloy include Ti—Ni alloys, Ti—Ni—Cu alloys, Ti—Ni—Fe alloys, Cu—Zn alloys, Cu—Zn—Al alloys, Cu—Al—Ni alloys, Cu—Au—Zn alloys, Cu—Sn alloys, Ni—Al alloys, Ag—Cd alloys, Au—Cd alloys, In—Tl alloys, and In—Cd alloys.

The use of such a superelastic alloy helps ensure that the deformable section 9 attains a sufficient flexibility and a restoring property with respect to deformation and that, even when the deformable section 9 is repeatedly brought into deformation (expansion/contraction), its excellent restoring property can prevent it from acquiring a substantially permanent or unremovable deformation (for example, remaining in an expanded state).

It is to be understood that the transformation temperature range of the superelastic alloy is not particularly limited. For example, the transformation temperature range is preferably −20 to 100□, more preferably −20 to 50□. This provides the advantage that the deformable section 9 is deformed more easily at an organism temperature.

The length of the deformable section 9 in its initial state is not limited to any particularly dimension. To provide an example, the length is preferably 5 to 100 mm, more preferably 10 to 70 mm.

The mean outside diameter of the deformable section 9 in its initial state is also not particularly limited. Providing an example, the mean outside diameter is preferably 2 to 20 mm, more preferably 4 to 10 mm.

The method of using the disclosed example of the catheter assembly 1A described above is as follows.

[1] First, the position of the atheroma 200 relative to a blood vessel is preliminarily confirmed under fluoroscopy. In addition, the catheter assembly 1A in its assembled state is preliminarily positioned in the condition in which the head section 5A protrudes from the distal end 211 of the outer catheter body 21 (the protruding condition or the condition shown in FIG. 1). While maintaining this condition, the guide wire 10 is inserted into the inner catheter 3A. The protruding condition of the head section 5A is securely maintained by the above-mentioned inserted condition maintaining means or fixing means.

[2] Next, only the guide wire 10 is inserted into the blood vessel through a sheath (not shown), and, further, the distal end of the guide wire 10 pierces (i.e., is inserted into) the atheroma 200. With the guide wire so placed, the catheter assembly 1A is advanced along the guide wire 10. Further, the head section 5A is inserted into the atheroma 200. Then, the fixed condition under the action of the inserted condition maintaining means is released, and the intermediate catheter 7, the inner catheter 3A and the guide wire 10 are collectively advanced (moved in the distal direction) relative to the outer catheter 2. This results in the head section 5A and the deformable section 9 being inserted in the atheroma 200 as shown in FIG. 2. These operations can be relatively easily carried out under fluoroscopy.

[3] Subsequently, in the condition shown in FIG. 2, the inner catheter 3A is reciprocated relative to the intermediate catheter 7 between the positions shown in FIGS. 2 and 3. By this operation, as above-mentioned, the atheroma 200 is stirred and made comparatively soft.

[4] Next, the inner catheter 3A is operated to return the deformable section 9 to its initial state or position. Thereafter, the intermediate catheter 7, the inner catheter 3A and the guide wire 10 are collectively pulled in the proximal direction relative to the outer catheter 2 as shown in FIG. 4. By this operation, as mentioned above, the atheroma 200 is gradually sucked into the outer catheter 2 as illustrated in FIG. 5. At last, the atheroma 200 is contained in the outer catheter 2 as depicted in FIG. 6.

[5] After it is confirmed that the atheroma 200 is contained in the outer catheter 2, the guide wire 10 is evulsed or withdrawn from the second lumen 52 as generally shown in FIG. 6. This results in the sucking function offered by the catheter assembly 1A disappearing or attenuating, whereby it is possible to generally avoid the situation in which, for example, blood is sucked into the outer catheter 2. After the evulsion of the guide wire 10, the catheter assembly 1A as a whole is evulsed or removed from the blood vessel while maintaining the contained condition of the atheroma 200. As a result, the atheroma 200 is removed from the inside of the blood vessel.

[6] A method may be also adopted in which the operation in [5] above is not conducted and, instead, the atheroma 200 is sucked further into the outside hub 4 and is removed (sucked or drawn in) via the branch section 47 connected to a sucking or drawing-in device such as a syringe.

An operation such as that described above in connection with one disclosed embodiment, results in the atheroma 200 in a blood vessel being assuredly captured and removed.

In addition, after the operation in [5] above, in a situation where it is desired that the catheter assembly 1A once again exhibits the sucking or drawing-in function, the guide wire 10 located in the first lumen 311 is again inserted into the second lumen 52. In this condition, the inner catheter 3A is pulled, whereby the sucking function is displayed assuredly.

In this disclosed embodiment of the catheter assembly 1A, depending on the inside diameter of the outer catheter body 21, the deformable section 9 in the expanded state can be contained in the outer catheter body 21. Where the catheter assembly 1A is configured in this manner, the atheroma 200 having entered (having been contained) into the deformable section 9 expanded by the operation in [3] above can be contained (captured) in the outer catheter body 21 together with the deformable section 9.

In addition, in the inner catheter 3A, the inside diameter of the inner catheter body 31 (first lumen 311) is set to be greater than the inside diameter of the head section 5A (second lumen 52). This helps ensure that the sucking function displayed by the catheter assembly 1A disappears or attenuates when the guide wire 10 inserted in the first lumen 311 and the second lumen 52 is only evulsed from the second lumen 52 without being completely evulsed from both lumens 52, 311.

In this manner, the insertion/non-insertion of the guide wire 10 in relation to the second lumen 52 can be selected. This makes it possible to select the closure/opening of the second lumen 52, and, therefore, to suck or draw in the atheroma 200 when the second lumen 52 is closed (plugged up), and to restrain the sucking function when the second lumen 52 is opened.

FIG. 9 illustrates a second embodiment of the catheter assembly. The following description will primarily center on the differences between this embodiment and the above-described embodiment. Features in this second embodiment similar to those in the first embodiment will not be described again in detail.

This embodiment is the same as the first embodiment above, except for the amount of protrusion of the deformable section from the distal end of the outer catheter body.

In the deformable section 9 of the catheter assembly 1A′ shown in FIG. 9, only a portion on the distal side of the deformable section 9 protrudes from the distal end 211 of the outer catheter body 21. Where the inner catheter 3A is operated under this condition, an intermediate part of the deformable section 9 is restricted by an edge part 213 on the inside of the distal end 211 of the outer catheter 2, so that the extent of expansion of the deformable section 9 is generally smaller than the extent of expansion of the deformable section 9 in the first embodiment above. That is, in the second embodiment, the portion of the deformable section 9 on the distal side of the deformable section 9 protrudes and is expanded. This makes it possible to deform the deformable section 9 according, for example, to the inside diameter of the blood vessel or the size of the atheroma 200, i.e., according to the individual case of disease.

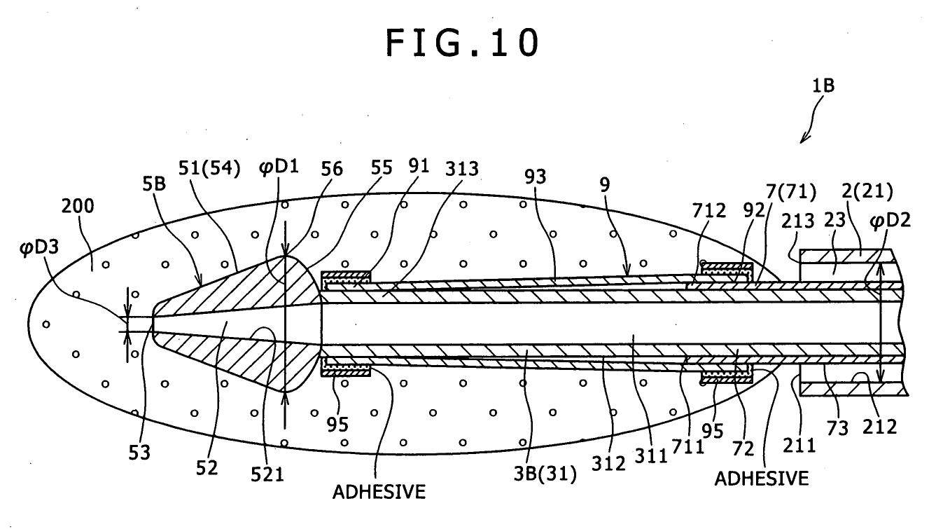

FIGS. 10-12 illustrate operating conditions of a catheter assembly according to a third embodiment. The following description centers primarily upon the differences between this embodiment and the above-described embodiments. A detailed description of features in this third version of the catheter assembly that are the same as or correspond to features in an earlier embodiment will not be repeated.

Generally speaking, this third embodiment is the same as the first embodiment described above, except for the size of the head section.

As shown in FIG. 10, in the catheter assembly 1B, the outside diameter φD1 of the maximum outside diameter part 56 of the head section 5B of the inner catheter 3B is greater, in its natural state, than the inside diameter φD2 of the outer catheter body 21.

In addition, the inside diameter of the head section 5B in its natural state gradually decreases along the distal direction. The minimum inside diameter φD3 of the head section 5B is approximately equal to or slightly smaller than the outer diameter of the guide wire 10. This helps ensure that, when the guide wire 10 is inserted in the second lumen 52 (head section 5B), the second lumen 52 is plugged up or closed off by the guide wire 10.

As shown in FIGS. 10-12, when the sucking operation is conducted in this embodiment, the guide wire 10 may have been previously evulsed from the catheter assembly 1B (second lumen 52). The reason will be described below.

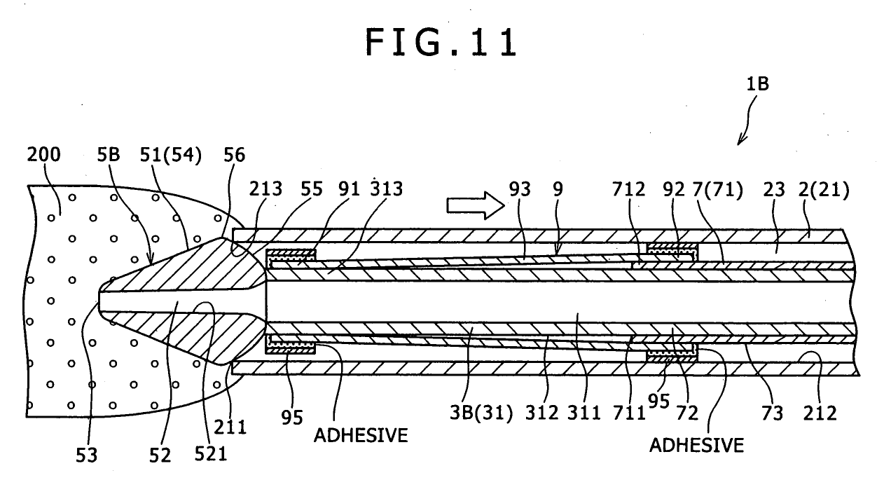

In the catheter assembly 1B, by moving the intermediate catheter 7 and the inner catheter 3B collectively in the proximal direction relative to the outer catheter 2 starting from the condition or position shown in FIG. 10, the second taper section 55 of the head section 5B is first pressed by the edge part 213 on the inside of the distal end 211 of the outer catheter body 21 as shown in FIG. 11.

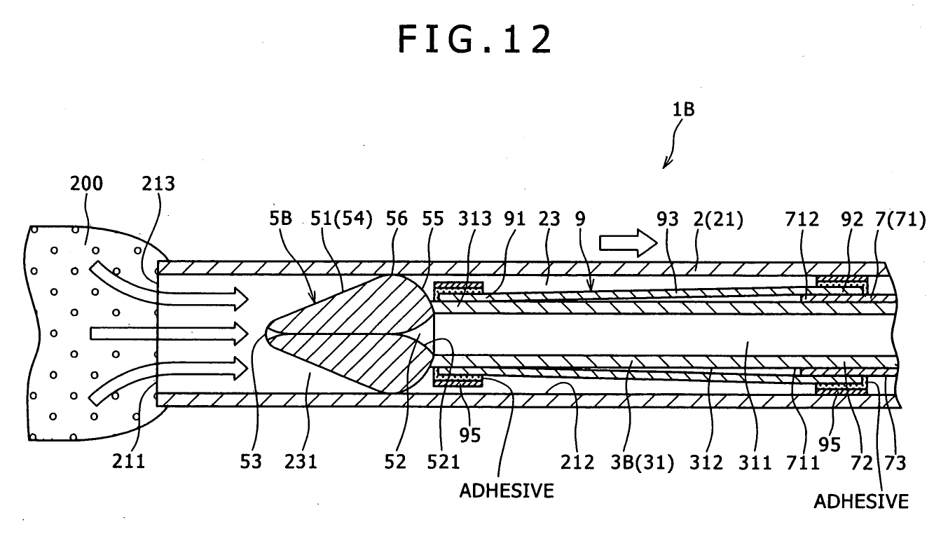

When the intermediate catheter 7 and the inner catheter 3B are collectively moved further in the proximal direction, the edge part 213 sequentially rides over (presses) the second taper section 55 and the maximum outside diameter part 56, and the maximum outside diameter part 56 slides inside the outer catheter body 21. At this time, as shown in FIG. 12, the maximum outside diameter part 56 (outer peripheral surface 51) is pressed inwardly by the inner peripheral surface 212 of the outer catheter body 21, whereby different portions of the inner peripheral surface 521 of the head section 5B are put into close contact with each other. In other words, the second lumen 52 is closed, more specifically self-closed.

By collectively pulling the intermediate catheter 7 and the inner catheter 3B under the condition where the second lumen 52 is thus closed, the pressure inside the space 231 is reduced. As a result, the atheroma 200 stirred by the deformable section 9 is assuredly sucked or drawn (contained) into the pressure-reduced space 231.

Thus, in the catheter assembly 1B shown in FIGS. 10-12, the second lumen 52 self-closes (has a self-closing property), whereby substantially the same effect (sucking effect) as that in the condition where the guide wire 10 is inserted in the second lumen 52 to close (plug up) the second lumen 52 can be obtained. Thus, in this third embodiment, it is not necessary to maintain the guide wire in the second lumen 52 in order to achieve the sucking or drawing-in effect.

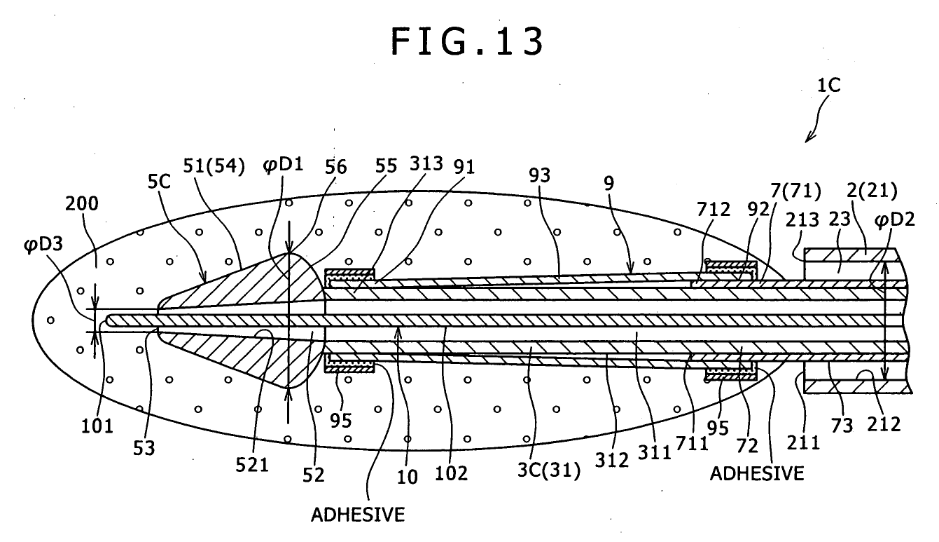

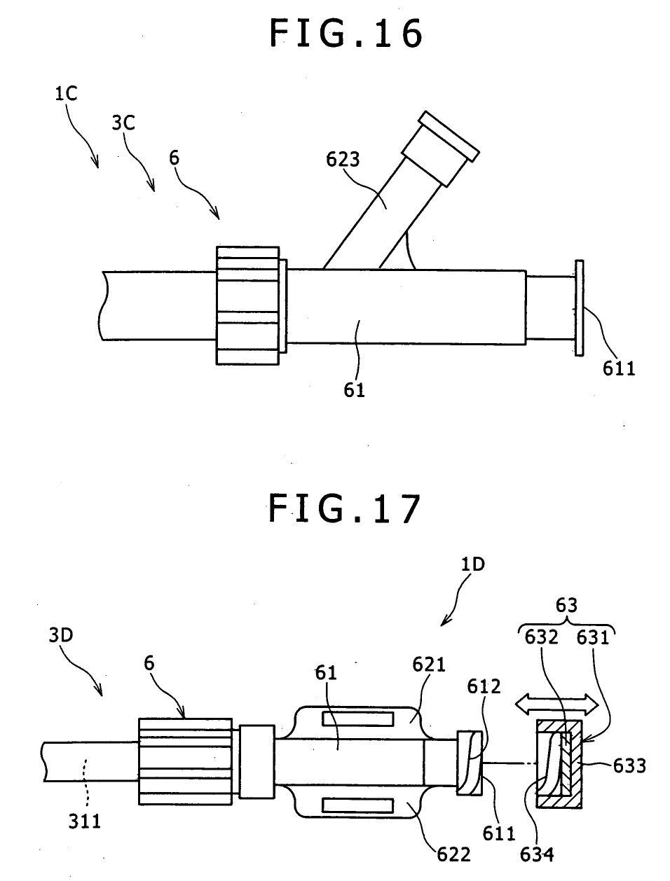

FIGS. 13-15 illustrate operating conditions of a catheter assembly according to a fourth embodiment, and FIG. 16 illustrates the vicinity of the inside hub of the catheter assembly. The following description centers primarily upon the differences between this embodiment and the above-described embodiments. A detailed description of features in this fourth embodiment of the catheter assembly that are the same as or correspond to features in an earlier embodiment will not be repeated.

This embodiment is the same as the third embodiment above, except for the inside diameter of the head section and the structure of the inside hub.

As shown in FIG. 13, in the catheter assembly 1C, the minimum inside diameter φD3 of the head section 5C of the inner catheter 3C is greater, in its natural state, than the outside diameter of the guide wire 10. That is, in the condition where the guide wire 10 is inserted in the head section 5C (second lumen 52) in the natural state, the head section 5C and the guide wire 10 are in a loose fit relation to each other.

In addition, as shown in FIG. 16, in the catheter assembly 1C, the inside hub 6 includes a tubular inside hub body 61 and a branch section 623 branched from an intermediate part of the inside hub body 61.

This helps ensure that, even in the condition where the guide wire 10 is inserted in the head section 5C as shown in FIG. 13, it is possible, for example, to supply (dose) a liquid medicine (drug) into the blood vessel through the branch section 623 and the inner catheter 3C (and via the distal end 53 of the head section 5C).

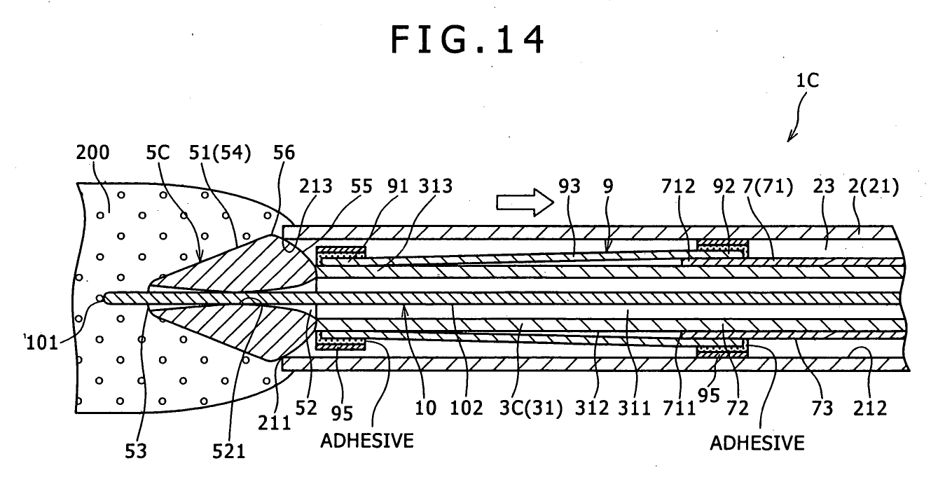

In the catheter assembly 1C, when the intermediate catheter 7 and the inner catheter 3C and the guide wire 10 are collectively moved in the proximal direction relative to the outer catheter 2 starting from the condition shown in FIG. 13, the second taper section 55 of the head section 5C is first pressed by the edge part 213 on the inside of the distal end 211 of the outer catheter body 21, as shown in FIG. 14. At this time, in the head section 5C, the inner peripheral surface 521 of the second lumen 52 approaches (or comes into contact with) the outer peripheral surface 102 of the guide wire 10. In other words, the second lumen 52 starts becoming constricted.

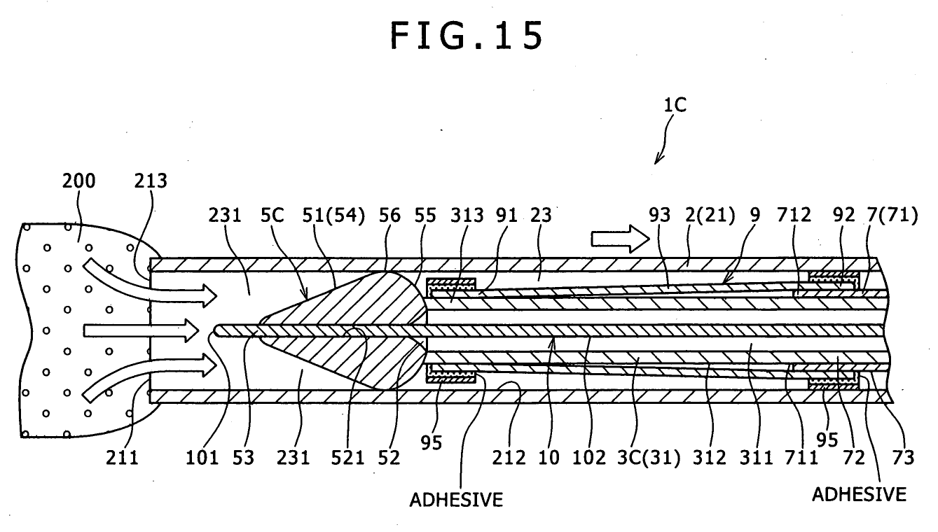

When the intermediate catheter 7, the inner catheter 3C and the guide wire 10 are collectively moved further in the proximal direction, the edge part 213 sequentially rides over (presses) the second taper section 55 and the maximum outside diameter part 56, and the maximum outside diameter part 56 slides inside the outer catheter body 21. At this time, as shown in FIG. 15, the maximum outside diameter part 56 (outer peripheral surface 51) is pressed by the inner peripheral surface 212 of the outer catheter body 21, whereby the second lumen 52 is constricted. With the second lumen 52 thus constricted, the inner peripheral surface 521 makes close contact with the outer peripheral surface 102 of the guide wire 10. As a result, the second lumen 52 is closed (plugged up).

By pulling collectively the intermediate catheter 7 and the inner catheter 3C and the guide wire 10 under the condition where the second lumen 52 is thus closed (plugged up), the pressure inside the space 231 is reduced. As a result, the atheroma 200 stirred by the deformable section 9 is assuredly sucked or drawn (contained) into the pressure-reduced space 231.

FIG. 17 is a partly longitudinally cross-sectional view showing the vicinity of the inside hub of a catheter assembly according to a fifth embodiment. The following description will primarily center on the differences of this embodiment from the above-described embodiments. A description of features in this version similar to those in the embodiments described above is not repeated. This embodiment is the same as the first embodiment described above, except that the inner catheter further includes a cap.

In the catheter assembly 1D shown in FIG. 17, the inner catheter 3D has a cap 63. The cap 63 is detachably mounted to the proximal end opening 611 of the inside hub 6.

The cap 63 includes a bottomed tubular cap body 631, and a packing (sealing member) 632 disposed at the bottom part 633 of the cap body 631.

The inner peripheral surface of the cap body 631 is formed with a female screw (thread) 634. A male screw (thread) 612 formed on the outer periphery of the proximal end of the inside hub 6 is screw (threadably) engaged with the female screw 634. By the screw engagement between these screws (threaded parts), the cap 63 is mounted to the inside hub 6.

The material forming the cap body 631 is not limited to a specific material. As examples, various metallic materials and various plastics and the like can be used either singly or in combination.

The packing 632 is a plate-like body formed of any of various rubber materials, for example.

In the condition where the cap 63 thus configured is mounted to the proximal end opening 611, the packing 632 is clamped between the bottom part 633 of the cap body 631 and the proximal end opening 611 of the inside hub 6. As a result, the proximal end opening 611 (the proximal end of the first lumen 311) is sealed up, i.e., closed.

In this embodiment, even if the guide wire 10 has been evulsed or removed from the catheter assembly 1D (second lumen 52) at the time of the sucking operation, the sealing of the proximal end opening 611 by the cap 63 helps ensure that the atheroma 200 stirred by the deformable section 9 is assuredly sucked into the outer catheter 2.

Thus, in the catheter assembly 1D, with the cap 63 mounted to the inside hub 6, substantially the same effect as that in the condition where the guide wire 10 is inserted in the second lumen 52 to close (plug up) the second lumen 52 can be obtained.

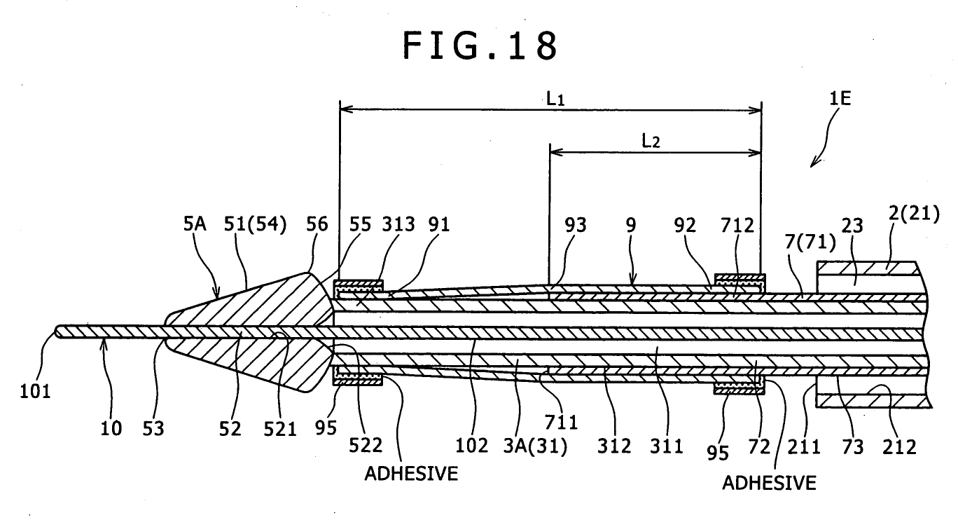

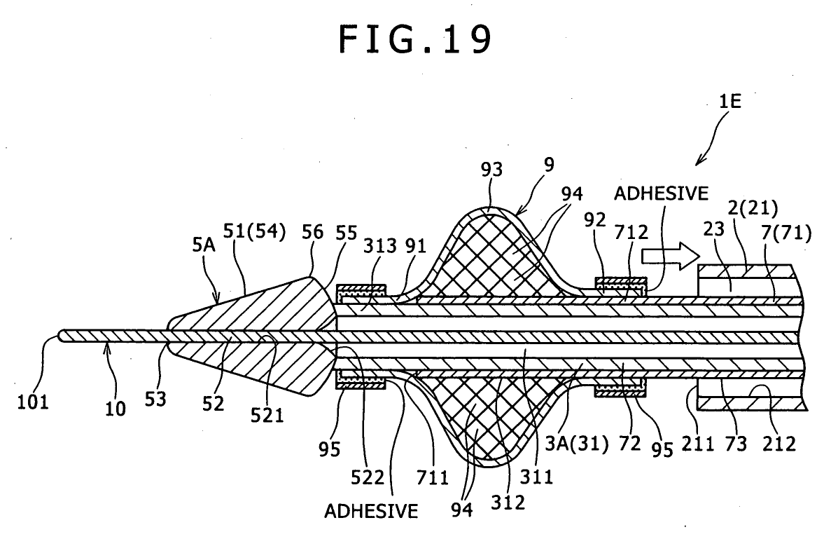

FIGS. 18 and 19 illustrate a sixth embodiment of the catheter assembly, with FIG. 18 showing a contracted state of the deformable section and FIG. 19 showing a maximally expanded state of the deformable section 9.

The following description will primarily center on differences of this embodiment relative to the above-described embodiments. Features in this second embodiment similar to those in the first embodiment will not be described again in detail.

This embodiment is the same as the first embodiment above, except that this catheter assembly further includes expansion amount restricting means.

In the catheter assembly 1E shown in FIGS. 18 and 19, the distal end 91 of the deformable section 9 is joined to the distal end 313 of the inner catheter body 31, and the proximal end 92 of the deformable section 9 is joined to an intermediate part of the intermediate catheter body 71. That is, the distal end 313 of the inner catheter body 31 and the intermediate part of the intermediate catheter body 71 are coupled to each other through the deformable section 9. This ensures that the distal end 711 of the intermediate catheter body 71 is located at an intermediate part in the longitudinal direction of the deformable section 9, i.e., in the vicinity of a middle part 93 of the deformable section 9 in its contracted state, in the configuration shown in FIG. 18.

When the inner catheter 3A is pulled in the proximal direction starting from the condition shown in FIG. 18, the distal end 91 of the deformable section 9 approaches the distal end 711 of the intermediate catheter body 71 while the deformable section 9 is being expanded by virtue of the pulling operation. Then, eventually, the vicinity of the distal end 91 of the inner peripheral surface of the deformable section 9 comes into contact with the distal end 711 of the intermediate catheter body 71, as shown in FIG. 19. This prevents the inner catheter 3A from being further pulled in the proximal direction from the condition shown in FIG. 19. It is thus possible to securely prevent the deformable section 9 from excessive expansion. If the deformable section 9 is expanded excessively, a situation may result in which, for example, when it is attempted to again contract the expanded deformable section 9, the deformable section 9 would not be contracted easily. In the catheter assembly 1E, on the contrary, such a potential difficulty can be inhibited or prevented because the deformable section 9 is restrained from excessive expansion.

Thus, in the catheter assembly 1E, the distal end 711 of the intermediate catheter body 71 located at an intermediate part in the longitudinal direction of the deformable section 9 is an example of expansion amount restricting means for restricting the maximum amount of expansion of the deformable section 9.

Incidentally, the length of the distance L₂ of the distal end 711 of the intermediate catheter body 71 from the proximal end of the deformable section 9 in the contracted state is not particularly limited. For example, the distance L₂ is preferably 0.3 to 0.7 times the overall length L₁ of the deformable section 9 in the contracted state, more preferably 0.4 to 0.6 times the overall length L₁.

Besides, in the catheter assembly 1E, the maximum amount of expansion of the deformable section 9 is appropriately set according to the distance L₂.

In addition, as has been described in the first embodiment above, the deformable section 9 is formed by combining a plurality of wires, composed of an alloy exhibiting superelasticity, in a net-like form. In this embodiment, wires formed of a material having radiopacity are further knitted into the deformable section 9. This desirably imparts radiopacity characteristics to the deformable section 9, and so the deformed state (contracted state and expanded state) of the deformable section 9 can be checked or confirmed under fluoroscopy.

Incidentally, the material for imparting radiopacity is not limited to any particular material. Examples of suitable materials include platinum, gold, tungsten, tantalum, iridium and alloys thereof.

Besides, the number of the radiopaque wires disposed is set at such a level that the deformable section 9 can be deformed easily and securely. For example, the number of the radiopaque wires may be set in the range of 5 to 50% based on the number of the superelastic wires.

In addition, in the case where the radiopaque wires are comparatively small in wire diameter, a plurality (e.g., two or three) of the wires may be stranded to obtain a larger overall wire diameter. By this, it is possible to enhance the radiopacity of the deformable section 9.

The method of imparting radiopacity to the deformable section 9 is not limited to radiopaque wires being knitted into the deformable section 9. For example, another method involves coating the deformable section 9 with a radiopaque material.

As has been described in the first to sixth embodiments above, the catheter assembly disclosed here may be embodied in one form in which the head section alone (the head section itself) functions as a sucking body and in another form in which the head section and the guide wire inserted in the head section function as a sucking body.

The form of the catheter assembly in which the head section alone functions as the sucking body includes the catheter assembly in which the head section self-closes as in the third embodiment, and the catheter assembly in which the head section is solid as in the first embodiment (also in the fifth embodiment and in the sixth embodiment).

On the other hand, the form of the catheter assembly mode in which the head section and the guide wire operate together to function as the sucking body includes the catheter assembly in which the second lumen of the head section is closed (plugged up) with the guide wire in the natural condition (normally) as in the first embodiment, and the catheter assembly in which the head section and the guide wire are in loose fit in the natural condition but the pressing of the head section by the inner peripheral surface of the outer catheter clears the loose fit condition so as to close the second lumen as in the fourth embodiment.

While the catheter assembly according to the various versions is described above with reference to various embodiments shown in the drawings, the catheter assembly is not limited in that regard as components and features of the catheter assembly can be replaced by other components or features exhibiting functions the same as or similar/equivalent to those described above. Further, arbitrary components or structures may be added to the above-described components.

In addition, the catheter assembly described here may be a combination of two or more of the configurations (characteristic features) of the above-described embodiments.

For example, the inside hub in the third embodiment and the fourth embodiment may be so configured that a cap substantially the same as that in the fifth embodiment can be mounted thereto.

Further, also in the third to fifth embodiment, the deformable section may be deformed in the state of protruding beginning with its intermediate part as in the second embodiment.

As discussed above, the catheter assembly comprises the outer catheter body 21, the intermediate catheter body 71 and the inner catheter body 31. The terms outer, intermediate and inner are used in the context of describing the different catheter bodies relative to one another (e.g., the intermediate catheter body is an intermediate catheter body relative to the inner and outer catheter bodies, and the inner catheter body is an inner catheter body relative to the outer and intermediate catheter bodies).

The principles, preferred embodiments and other disclosed aspects have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A catheter assembly to be inserted into an organism to capture foreign matter present in the organism, comprising:
an elongated tubular outer catheter body possessing an open distal end;
an elongated tubular intermediate catheter body positioned inside the outer catheter body and possessing an open distal end;
an elongated inner filamentous element positioned inside the intermediate catheter body, the inner filamentous element possessing a distal part;
an elastic head section at the distal part of the inside filamentous element and connected to the inner filamentous element, the elastic head section protruding distally beyond the open distal end of the intermediate catheter body; and
stirring means insertable into the foreign matter for stirring the foreign matter as the stirring means protrudes distally beyond the distal open end of the outer catheter body;
wherein after stirring by the stirring means, the intermediate catheter body and the inner filamentous element are collectively moved in a proximal direction relative to the outer catheter body to move the head section in the proximal direction into the outer catheter body to suck the stirred foreign matter through the distal open end of the outer catheter body and into the outer catheter body.

2. The catheter assembly as set forth in claim 1, wherein said stirring means comprises a deformable section which connects the distal part of the inner filamentous element and a part of the intermediate catheter body to each other and which is expanded/contracted when the distal part of the inside filamentous element and the distal part of the intermediate catheter body are moved toward/away from each other.

3. The catheter assembly as set forth in claim 2, wherein the deformable section is comprised of a tube.

4. The catheter assembly as set forth in claim 3, wherein the tube is provided with a multiplicity of holes penetrating a wall part of the tube.

5. The catheter assembly as set forth in claim 2, wherein an extent or location of the expansion of the deformable section varies depending on an amount of protrusion of the deformable section from the open distal end of the outer catheter body.

6. The catheter assembly as set forth in claim 2, wherein the deformable section is constructed to contain the foreign matter in the deformable section upon being expanded.

7. The catheter assembly as set forth in claim 2, wherein the deformable section is comprised of an alloy which exhibits superelasticity in the organism.

8. The catheter assembly as set forth in claim 2, wherein the deformable section is radiopaque.

9. The catheter assembly as set forth in claim 1,
wherein the inner filamentous element possesses a first lumen extending along a longitudinal direction of the filamentous element, and
the head section is formed with a second lumen communicating with said first lumen and open at a distal end of the head section.

10. The catheter assembly as set forth in claim 9, wherein the first lumen and the second lumen are each adapted to receive a guide wire.

11. The catheter assembly as set forth in claim 10, wherein the second lumen is closed by the guide wire when the guide wire is inserted therein to block passage of fluid through the second lumen.

12. The catheter assembly as set forth in claim 9, wherein the head section possesses a maximum outside diameter in its natural state in which the head section protrudes distally beyond the open distal end of the outer catheter body that is dimensioned so that when the head section is drawn into the outer catheter body a reduction in pressure arises in a space in the outer catheter body that is distal of the maximum outside diameter.

13. The catheter assembly as set forth in claim 9,
wherein the head section possesses a maximum outside diameter in its natural state in which the head section protrudes distally beyond the open distal end of the outer catheter body that is greater than an inside diameter of the outer catheter body.

14. The catheter assembly as set forth in claim 13, wherein when the head section slides in the outer catheter body, an outer peripheral surface of the head section is pressed by an inner peripheral surface of the outer catheter body and causes the second lumen to be constricted or closed.

15. The catheter assembly as set forth in claim 9, further comprising an inside hub comprised of a tubular element which is joined to a proximal end of the inner filamentous element and which communicates with the first lumen.

16. The catheter assembly as set forth in claim 15, further comprising a cap detachably attached to a proximal end of the inside hub, a proximal end side of the first lumen being closed when the cap is attached to the proximal end of the inside hub.

17. The catheter assembly as set forth in claim 1, wherein an outer diameter of the inner filamentous element is smaller than a maximum outside diameter of the head section.

18. The catheter assembly as set forth in claim 1, further comprising inserted condition maintaining means for maintaining a position of the intermediate catheter body in the outer catheter body.

19. The catheter assembly as set forth in claim 18, wherein said inserted condition maintaining means maintains a condition in which the stirring means protrudes by a predetermined amount distally beyond the distal open end of the outer catheter body.

20. A catheter assembly to be inserted into an organism to capture foreign matter present in the organism, comprising:
an elongated tubular outer catheter body possessing a lumen opening at a distal end of the outer catheter body;
an elongated tubular intermediate catheter body positioned in the lumen of the outer catheter body and possessing a lumen opening at a distal end of the intermediate catheter body, the intermediate catheter being longitudinally movable relative to the outer catheter body;
an elongated tubular inner catheter body positioned in the lumen of the intermediate catheter body, the inner catheter being longitudinally movable relative to the outer catheter body;
a tube connected to the intermediate catheter body and the inner catheter body at respective spaced apart locations, an intermediate portion of the tube between the two spaced apart locations comprising a deformable section that is positionable in the foreign matter and deformable upon application of a force to the deformable section; and
the inner catheter body and the intermediate catheter body being relatively longitudinally movable to apply a force to the deformable section of the tube positioned in the foreign matter to alternatively expand and contract the deformable section in a manner which stirs the foreign matter, with the stirred foreign matter being subsequently capturable in the outer catheter body.

21. The catheter assembly as set forth in claim 20, further comprising a head section connected to the intermediate catheter body to move together with the intermediate catheter body, the head section protruding distally beyond the distal end of the outer catheter body in a normal state of the head section, the head section being movable into the outer catheter body by longitudinal movement of the intermediate catheter body in a proximal direction relative to the outer catheter body.

22. The catheter assembly as set forth in claim 21, wherein the head section possesses a maximum outer dimension portion which engages an inner surface of the lumen in the outer catheter when the head section is moved into the lumen of the outer catheter.

23. The catheter assembly as set forth in claim 20, wherein the tube is connected to a distal end of the inner catheter body at one of the spaced apart locations and is connected to a distal end of the intermediate catheter body at the other of the two spaced apart locations.

24. The catheter assembly as set forth in claim 20, wherein the head section comprises a lumen extending throughout the head section.

25. The catheter assembly as set forth in claim 20, wherein the tube comprises a plurality of holes which permit the foreign matter to enter an interior of the deformable section as the deformable section is deformed.

26. A catheter assembly to be inserted into an organism to capture foreign matter present in the organism, comprising:
an elongated tubular outer catheter body possessing a lumen opening at a distal end of the outer catheter body, the lumen possessing a diameter and defining an inner surface of the outer catheter body;
an elongated tubular intermediate catheter body positioned in the lumen of the outer catheter body and possessing a lumen opening at a distal end of the intermediate catheter body, the intermediate catheter being longitudinally movable relative to the outer catheter body;
an elongated tubular inner catheter body positioned in the lumen of the intermediate catheter body, the inner catheter being longitudinally movable relative to the outer catheter body;
a head section connected to the intermediate catheter body to move together with the intermediate catheter body, the head section being comprised of elastic material and protruding distally beyond the distal end of the outer catheter body in a normal state of the head section, the head section being movable into the outer catheter body by longitudinal movement of the intermediate catheter body in a proximal direction relative to the outer catheter body; and
the head section possessing a maximum outer dimension portion at which an outer dimension of the head section is greatest, the maximum outer dimension portion of the head section being structurally sized relative to the diameter of the lumen in the outer catheter body such that when the head section is moved into the lumen of the outer catheter body from the normal state as a result of longitudinal movement of the intermediate catheter body in the proximal direction relative to the outer catheter body, the maximum outer dimension portion of the head section engages the inner surface of the outer catheter body and creates a space of reduced pressure in the lumen of the outer catheter body distally of the head section to draw the foreign matter into the lumen of the outer catheter body.

27. The catheter assembly as set forth in claim 26, wherein the head section comprises a lumen.

28. The catheter assembly as set forth in claim 27, wherein the maximum outer dimension portion of the head section is structurally sized relative to the diameter of the lumen in the outer catheter body such that when the head section is moved into the lumen of the outer catheter body, the head section is compressed to construct or close the lumen in the head section.

29. A method of removing foreign matter present in an organism, comprising:
inserting a catheter assembly into an organism, the catheter assembly comprising:
an elongated tubular outer catheter body;
an elongated tubular intermediate catheter body positioned in the outer catheter body;
an elongated tubular inner catheter body positioned in the intermediate catheter body;
a tube connected to the intermediate catheter body and the inner catheter body at respective spaced apart locations;
moving the catheter assembly within the organism to position at least a portion of the tube in the foreign matter;
stirring the foreign matter by effecting relative longitudinal movement between the intermediate catheter body and the inner catheter body to cause a deformable section of the tube located between the two spaced apart locations to deform in the foreign matter;
removing the foreign matter through the outer catheter body.

30. The method as set forth in claim 29, wherein the catheter assembly is moved within the organism to position at least a portion of the tube in the foreign matter by advancing the catheter assembly over a guide wire having a distal end positioned in the foreign matter, and wherein the deformable section is deformed while the distal end of the guide wire remains in the foreign matter.

31. The method as set forth in claim 29, wherein the deformation of the deformable section comprises repeatedly expanding and contracting the deformable section.

32. The method as set forth in claim 29, wherein the deformable section comprises a plurality of through holes passing through a wall of the tube, and during deformation of the deformable section the foreign matter flows into the tube through the through holes.

33. A method of removing foreign matter present in an organism, comprising:
inserting a catheter assembly into an organism, the catheter assembly comprising:
an elongated tubular outer catheter body;
an elongated tubular intermediate catheter body positioned in the outer catheter body;
an elongated tubular inner catheter body positioned in the intermediate catheter body;
a head section connected to the intermediate catheter body to move together with the intermediate catheter body and protruding distally beyond a distal end of the outer catheter body in a normal state of the head section;
moving the catheter assembly within the organism to position the head section in the foreign matter;
moving the intermediate catheter body in a proximal direction relative to the outer catheter body to move the head section into the outer catheter body so that an outer surface of the head section engages an inner surface of the outer catheter body to create a space of reduced pressure in the outer catheter body distally of the head section that draws the foreign matter into the outer catheter body.

34. The method as set forth in claim 33, wherein the catheter assembly is moved within the organism to position the head section in the foreign matter by advancing the catheter assembly over a guide wire that extends through a lumen in the head section, and wherein the head section is moved into the outer catheter body while the guide wire remains in the lumen of the head section.

35. The method as set forth in claim 34, further comprising withdrawing the guide wire from the lumen of the head section after at least some of the foreign matter has been drawn into the space of reduced pressure in the outer body so that the foreign matter flows through the lumen of the head section.

36. The method as set forth in claim 33, wherein the catheter assembly is moved within the organism to position the head section in the foreign matter by advancing the catheter assembly over a guide wire that extends through a lumen in the head section, and further comprising withdrawing the guide wire from the lumen of the head section before the head section is moved into the outer catheter body.

37. The method as set forth in claim 36, wherein after the guide wire has been withdrawn from the lumen of the head section, the lumen in the head section is constricted or closed as the head section is moved into outer catheter body.
